SEQUENCE LISTING (8 sequences)

SEQ ID NO: 1 - DNA, 1530 bp, Clostridium bolteae
atgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 2 - DNA, 1522 bp, Anaerotruncus colihominis
tcaaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 3 - DNA, 1529 bp, Ruminococcus torques
tacgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcgtccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctccttt

SEQ ID NO: 4 - DNA, 1527 bp, Clostridium symbiosum
atgagagtttgatcctggctcaggatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctccttt

SEQ ID NO: 5 - DNA, 1531 bp, Blautia producta
atcagagagtttgatcctggctcaggatgaacgctggcggcgtgcttaacacatgcaagtcgagcgaagcacttaagtggatctcttcggattgaagcttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggagggagctgccgaaggcgggaccgataactggggtgaagtcgtaacaaggtagccgtatcggaaggtgcggctggatcacctccttt

SEQ ID NO: 6 - DNA, 1529 bp, Dorea longicatena
aacgagagtttgatcctggctcaggatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 7 - DNA, 1537 bp, Erysipelotrichaceae bacterium
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcggccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccgatgactggggttaagtcgtaacaaggtatccctacgggaacgtggggatggatcacctccttt

SEQ ID NO: 8 - DNA, 1530 bp, Subdoligranulum spp
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

(19) United States
(12) Patent Application Publication
(10) Pub. No.: US 2022/0389369 A1
(43) Pub. Date: Dec. 8, 2022

(54) METHODS AND COMPOSITIONS FOR PRESERVING BACTERIA

(71) Applicant: Vedanta Biosciences, Inc., Cambridge, MA (US)

(72) Inventor: Scott Michonski, Belmont, MA (US)

(73) Assignee: Vedanta Biosciences, Inc., Cambridge, MA (US)

(21) Appl. No.: 17/760,674

(22) PCT Filed: Sep. 15, 2020
(86) PCT No.: PCT/US2020/050868

§ 371 Date: Mar. 15, 2022

Related U.S. Application Data
(60) Provisional application No. 62/901,205, filed on Sep. 16, 2019.

(51) Int. Cl. C12N 1/04; C12N 1/20

(52) U.S. Cl. CPC C12N 1/04; C12N 1/20

(57) ABSTRACT

The disclosure provides methods and compositions for the preservation of bacteria. Aspects of the present disclosure provide methods of preparing a preserved bacterial composition comprising flash freezing a bacterial composition and lyophilizing the flash frozen bacterial composition to produce a preserved bacterial composition. In some embodiments, the bacterial composition comprises one or more bacterial strains. In some embodiments, the one or more bacterial strains comprise one or more anaerobic bacterial strains. In some embodiments, the anaerobic bacterial strains are strict anaerobic bacteria.

### RELATED APPLICATION

This application claims the benefit under 35 U.S.C. § 119(e) of U.S. provisional application No. 62/901,205, filed Sep. 16, 2019. The entire contents of the referenced application are incorporated by reference herein.

### FIELD OF THE INVENTION

The disclosure provides methods and compositions for the preservation of bacteria.

### BACKGROUND

The human intestinal microbiome includes a large number of microorganisms. A significant number of these microorganisms are anaerobic bacteria. Compositions that include anaerobic bacteria that originated from the human intestinal microbiome have shown potential in the treatment of human disease (See e.g., Atarashi et al., Nature 500, 232, 2013; Atarashi et al., Cell 163, 1, 2015; Mathewson et al., Nature Immunology 17, 505, 2016). Anaerobic bacteria are challenging to preserve because of their sensitivity to oxygen. Improved compositions and methods for the preservation of anaerobic bacteria are needed therefore.

### SUMMARY

Aspects of the present disclosure provide methods of preparing a preserved bacterial composition comprising flash freezing a bacterial composition and lyophilizing the flash frozen bacterial composition to produce a preserved bacterial composition. In some embodiments, the bacterial composition comprises one or more bacterial strains. In some embodiments, the one or more bacterial strains comprise one or more anaerobic bacterial strains. In some embodiments, the anaerobic bacterial strains are strict anaerobic bacteria.

In some embodiments, the bacterial composition comprises one or more bacterial strains belonging to the class Clostridia. In some embodiments, the one or more bacterial strains belong to the family Clostridiaceae. In some embodiments, the bacteria comprise one or more bacterial strains belonging to the genus Clostridium. In some embodiments, the bacterial composition comprises one or more bacterial strains selected from the group consisting of Clostridium bolteae, Anaerotruncus colihominis, Eubacteriaum fissicatena, Clostridium symbiosum, Blautia producta, Dorea longicatena, Clostridium innocuum, and Flavinofractor plautti. In some embodiments, the bacterial composition comprises one or more bacterial strains comprising 16S rDNA sequences having at least 97% sequence identity with the nucleic acid sequences selected from the group consisting of SEQ ID NOs: 1-8.

In some embodiments, the method further comprises culturing the bacterial composition. In some embodiments, the bacterial composition is washed and resuspended in a formulation buffer. In some embodiments, the flash freezing is performed by contacting the bacterial composition with a super-cooled surface. In some embodiments, the flash freezing is performed by contacting the bacterial composition with liquid nitrogen.

In some embodiments, the bacterial composition has a symmetrical shape. In some embodiments, the symmetrical shape is a symmetrical frozen droplet. In some embodiments, the preserved bacterial composition is subjected to a temperature of −80° C.

In some embodiments, the lyophilizing comprises a primary drying step and a secondary drying step. In some embodiments, the primary drying step comprises subjecting the flash frozen bacterial composition to a temperature of −10° C. and under a pressure of 70 mTorr. In some embodiments, the secondary drying step comprises subjecting the flash frozen bacterial composition to a temperature of +20° C. and under a pressure of 70 mTorr.

In some embodiments, the method further comprises determining a level of viability in the preserved bacterial composition after lyophilizing. In some embodiments, the level of viability in the preserved bacterial composition is at least 10%, at least 15%, at least 20%, at least 25%, at least 30%, at least 35%, or at least 40% of colony forming units of the bacteria over a period of time. In some embodiments, the period of time is at least 1 week, at least 2 weeks, at least 4 weeks, at least 2 months, at least 3 months, at least 6 months, or at least 1 year or more.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWING

The accompanying drawing is not intended to be drawn to scale. The FIGURE is illustrative only and is not required for enablement of the disclosure. For purposes of clarity, not every component may be labeled in the drawing. In the drawing:

FIG. 1 shows the post-lyophilization viability of the indicated bacterial strains. The percent post-lyophilization viability (% viability on the y-axis) is calculated based on the enumerated colony forming units following lyophilization relative to the enumerated colony forming units following harvest of the bacterial culture. For each of the indicated bacterial strains, the left column corresponds to viability following the standard freezing process (i.e., freeze-drying tray) prior to lyophilization, and the right column corresponds to viability following the flash freezing droplets and lyophilizing methods described herein.

### DETAILED DESCRIPTION

The preservation of bacterial compositions, including anaerobic bacteria, has been challenging. While bacteria can be frozen down and regrown on plates or in solution, it has been difficult to standardize this process. There is a need to preserve bacteria that can be used for therapeutic purposes. Preservation processes, such as cryopreservation and lyophilization, have been well established for aerobic bacteria, and many factors that affect survival and recovery of aerobic bacteria in the preservation process are understood (Prakash et al. FEMS Microbiol Lett (2013)339:1-9). For bacterial products that rely on viable bacteria, enhancing the level of viability of bacteria following preservation processes have the potential to reduce costs associated with production of such products. Given that bacteria, such as anaerobic bacterial strains obtained from the human intestinal microbiome have shown potential in the treatment of human disease, improved methods for preserving bacteria that allow for high levels of bacterial recovery are needed.

Described herein are methods of preserving bacterial compositions comprising flash freezing a bacterial composition and lyophilizing the flash frozen bacterial composition to produce a preserved bacterial composition. The ability to freeze and store the flash frozen bacterial compositions allows greater flexibility with the preservation process. In some embodiments, the methods described herein are used for the preservation of anaerobic bacteria.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Provided herein are compositions and methods for the preservation of bacteria. In one aspect, the methods provided herein allow for preservation of bacterial compositions. In some embodiments, the methods described herein are used for the preservation of anaerobic bacteria. The compositions allow the bacteria to go through a freeze-dry cycle with minimal loss to viability. In some embodiments, the bacterial composition includes bacteria. In some embodiments, the bacterial composition includes one or more bacterial strains. In some embodiments, the bacterial composition includes a single bacterial strain. In some embodiments, the bacterial composition comprises at least 2, at least 3, at least 4, at least 5, at least 6, at least 7, or at least 8 or more bacterial strains (e.g., purified bacterial strains).

In some embodiments, the bacterial composition comprises one or more anaerobic bacterial strains (e.g., strict anaerobic bacteria). In some embodiments of the compositions provided herein, the anaerobic bacteria are strict anaerobic bacteria.

In some embodiments, the bacterial composition comprises one or bacterial strains belonging to the class Clostridia. In some embodiments, one or more bacterial strains are from the family Clostridiaceae. In some embodiments, the bacteria are from the genus Clostridium. In some embodiments, the bacteria belong to Clostridium cluster IV, XIVa, XVI, XVII, or XVIII. In some embodiments, the bacteria belong to Clostridium cluster IV, XIVa, or XVII. In some embodiments, the bacteria belong to Clostridium cluster IV or XIVa.

In some embodiments, the bacterial composition includes one or more of the following bacterial strains: Clostridium bolteae, Anaerotruncus colihominis, Sellimonas intestinalis, Clostridium symbiosum, Blautia producta, Dorea longicatena, Erysipelotrichaceae bacterium and Flavinofractor plautii. Bacterial strains Clostridium bolteae, Anaerotruncus colihominis, Sellimonas intestinalis, Clostridium symbiosum, Blautia producta, Dorea longicatena, Erysipelotrichaceae bacterium and Flavinofractor plautii are described, for instance, in PCT Publication No. WO 2017/218680, which is incorporated by reference in its entirety. The strains are also depicted in Table 1. It should be appreciated that alternative strain names, e.g., as depicted in Table 1, may be used as well.

In some embodiments, the bacterial composition includes one or more of the following bacterial strains: Clostridium bolteae 90A9, Anaerotruncus colihominis DSM17241, Sellimonas intestinalis, Clostridium bolteae, Anaerotruncus colihominis, Sellimonas intestinalis, Clostridium symbiosum, Blautia producta, Dorea longicatena, Erysipelotrichaceae bacterium and Flavinofractor plautii. In some embodiments, the bacterial composition includes two or more (e.g., 2, 3, 4, 5 6, 7, or 8) of the following bacterial strains: Clostridium bolteae, Anaerotruncus colihominis, Sellimonas intestinalis, Clostridium symbiosum, Blautia producta, Dorea longicatena, Erysipelotrichaceae bacterium and Flavinofractor plautii. In some embodiments, the bacterial composition includes Clostridium bolteae. In some embodiments, the bacterial composition includes Anaerotruncus colihominis. In some embodiments, the bacterial composition includes Sellimonas intestinalis. In some embodiments, the bacterial composition includes Clostridium symbiosum. In some embodiments, the bacterial composition includes Blautia producta. In some embodiments, the bacterial composition includes Dorea longicatena. In some embodiments, the bacterial composition includes Erysipelotrichaceae bacterium. In some embodiments, the bacterial composition includes Flavinofractor plautii.

In one aspect, as shown herein (e.g., in the Example) the methods provided herein allow for the preservation of anaerobic bacterial strains. Anaerobic strains that can be used in the methods of the current invention include bacterial strains that are used in therapeutic consortia, such as described for instance in PCT Publication Nos. WO2013/080561, WO2015/156419, WO2018/117263, WO2017/218680, WO2019/094837, and WO2019/118515. In one aspect, as shown herein (e.g., in the Example) the methods provided herein allow for the preservation of anaerobic bacterial strains belonging to Clostridium cluster IV, XIVa, or XVII. In one aspect, as shown herein (e.g., in the Example) the methods provided herein allow for the preservation of anaerobic bacterial strains Clostridium bolteae, Anaerotruncus colihominis, Sellimonas intestinalis, Clostridium symbiosum, Blautia producta, Dorea longicatena, Erysipelotrichaceae bacterium and Flavinofractor plautii. The exemplary bacterial strains can also be identified by their 16s rRNA sequences (SEQ ID NOs: 1-8). Identifying bacteria by their sequences furthermore allows for the identification of additional bacterial strains that are identical or highly similar to the exemplified bacteria. For instance, the 16s rRNA sequences of bacterial strains were used to identify the closest relative (based on percent identity) through whole genome sequencing and by comparing these sequences with 16S databases (Table 1). In addition, based on whole genome sequencing and comparing of the whole genome to whole genome databases, the bacterial strains having 16S rRNA sequences provided by SEQ ID NOs: 1-8 are most closely related to the following bacterial species: Clostridium bolteae 90A9, Anaerotruncus colihominis DSM 17241, Dracourtella massiliensis GD1, Clostridium symbiosum WAL-14163, Clostridium bacterium UC5.1-1D4, Dorea longicatena CAG:42, Erysipelotrichaceae bacterium 21_3, and Clostridium orbiscindens 1_3_50AFAA (see, e.g., Table 1). Thus, in one aspect it should be appreciated that each row of Table 1, the bacterial strains are highly similar and/or are identical. In some embodiments, in context of the instant disclosure the names of bacterial strains within a row of Table 1 can be used interchangeably.

TABLE 1
Examples of Bacterial species of the compositions disclosed herein

| | | | Closest species based on | | | |
|---|---|---|---|---|---|---|
| | | Closest species based on | Consensus SEQ ID # of 16S | Closest species based on | | |
| Strain | SEQ ID | Sanger sequencing of 16S | region as compared with 16S | WGS compared versus | Additional closely | Clostridium |
| number | NO: | region | database | WG databases | related sequences | cluster |
| VE303-1 | 1 | Clostridium bolteae | Clostridium bolteae | Clostridium bolteae 90A9 | | XIVa |
| VE303-2 | 2 | Anaerotruncus colihominis | Anaerotruncus colihominis | Anaerotruncus colihominis | | IV |
| | | | | DSM 17241 | | |
| VE303-3 | 3 | Eubacterium fissicatena | Dracourtella massiliensis | Dracourtella massiliensis | Ruminococcus | XIVa |
| | | | | GD1 | torques; Sellimonas | |
| | | | | | intestinalis | |
| VE303-4 | 4 | Clostridium symbiosum | Clostridium symbiosum | Clostridium symbiosum | | XIVa |
| | | | | WAL-14163 | | |
| VE303-5 | 5 | Blautia producta | Blautia producta | Clostridium bacterium | Blautia producta | XIVa |
| | | | | UC5.1-1D4 | ATCC 27340 | |
| VE303-6 | 6 | Dorea longicatena | Dorea longicatena | Dorea longicatena CAG:42 | | XIVa |
| VE303-7 | 7 | Clostridium innocuum | Clostridium innocuum | Erysipelotrichaceae | | XVII |
| | | | | bacterium 21_3 | | |
| VE303-8 | 8 | Flavinofractor plautii | Flavinofractor plautii | Clostridium orbiscindens | Subdolinogranulum | IV |
| | | | | 1_3_50AFAA | | |

Aspects of the disclosure relate to bacterial strains with 16S rDNA sequences that have sequence identity to a nucleic acid sequence of any one of the sequences of the bacterial strains or species described herein. The terms “identical,” percent “identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over an entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the bacterial composition includes one or more bacterial strain that has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9% or up to 100% sequence identity to any one of the strains or bacterial species described herein over a specified region or over the entire sequence. It would be appreciated by one of skill in the art that the term “sequence identity” or “percent sequence identity” in the context of two or more nucleic acid sequences or amino acid sequences, refers to a measure of similarity between two or more sequences or portion(s) thereof.

In some embodiments, the bacterial composition includes one or more bacterial strains, wherein the one or more bacterial strains include one or more 16s rDNA sequences having at least 97% sequence identity with nucleic acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the bacterial composition includes one bacterial strain, wherein the bacterial strain includes one or more 16s rDNA sequences having at least 97% sequence identity with nucleic acid sequences SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the bacterial composition includes one bacterial strain, wherein the bacterial strain includes one or more 16s rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

In some embodiments, the bacterial composition includes one bacterial strain, wherein the bacterial strain includes one or more 16s rDNA sequences having at least 97% sequence identity with nucleic acid sequences SEQ ID NO: 1. In some embodiments, the bacterial composition includes one bacterial strain, wherein the bacterial strain includes one or more 16s rDNA sequences having at least 97% sequence identity with nucleic acid sequences SEQ ID NO: 2. In some embodiments, the bacterial composition includes one bacterial strain, wherein the bacterial strain includes one or more 16s rDNA sequences having at least 97% sequence identity with nucleic acid sequences SEQ ID NO: 3. In some embodiments, the bacterial composition includes one bacterial strain, wherein the bacterial strain includes one or more 16s rDNA sequences having at least 97% sequence identity with nucleic acid sequences SEQ ID NO:4. In some embodiments, the bacterial composition includes one bacterial strain, wherein the bacterial strain includes one or more 16s rDNA sequences having at least 97% sequence identity with nucleic acid sequences SEQ ID NO:5. In some embodiments, the bacterial composition includes one bacterial strain, wherein the bacterial strain includes one or more 16s rDNA sequences having at least 97% sequence identity with nucleic acid sequences SEQ ID NO:6. In some embodiments, the bacterial composition includes one bacterial strain, wherein the bacterial strain includes one or more 16s rDNA sequences having at least 97% sequence identity with nucleic acid sequences SEQ ID NO:7. In some embodiments, the bacterial composition includes one bacterial strain, wherein the bacterial strain includes one or more 16s rDNA sequences having at least 97% sequence identity with nucleic acid sequences SEQ ID NO:8.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region of the nucleic acid or amino acid sequence or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, by computerized implementations of these algorithms (GAP, BESTFIT, FASTA, and TFASTA in the Wisconsin Genetics Software Package, Genetics Computer Group. Madison, Wis.), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402, and Altschul et al., J. Mol. Biol. (1990) 215:403-410, respectively.

It should be appreciated that the terms “bacteria” and “bacterial strains” as used herein are interchangeable.

As used herein, the term “isolated” refers to a bacteria or bacterial strain that has been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

In some embodiments, the bacterial strains are grown up from a single colony. In some embodiments, the bacterial strains are purified bacterial strains. As used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

The bacterial strains of the composition can be manufactured using fermentation techniques well known in the art. In some embodiments, the active ingredients are manufactured using anaerobic fermenters, which can support the rapid growth of anaerobic bacterial strains. The anaerobic fermenters may be, for example, stirred tank reactors or disposable wave bioreactors. Culture media such as BL media and EG media, or similar versions of these media devoid of animal components, can be used to support the growth of the bacterial species. In some embodiments, the bacterial composition is grown to a desired growth phase prior to flash freezing. In some embodiments, the bacterial composition is grown to a desired cell density prior to flash freezing. In some embodiments, the cell density is a desired optical density (e.g., OD₆₀₀) of the bacterial strain.

The bacterial product can be purified and concentrated from the fermentation broth by traditional techniques, such as centrifugation and filtration. Generally, the bacteria are pelleted prior to subjecting the bacterial composition to flash freezing. In some embodiments, the bacterial composition is washed prior to flash freezing. As used herein, the term “wash” or “washing” refers to series of steps to isolate bacterial cells and remove residual undesired components (e.g., cell debris, components of growth media). In some embodiments, the method involves isolating bacterial cells from a culture (e.g., growth media), resuspending the isolated bacterial cells in a wash buffer, and isolating the bacterial cells from the wash buffer by traditional techniques, such as centrifugation and filtration. In some embodiments, the isolated bacterial cells are resuspended in formulation buffer. In some embodiments, the method involves washing the bacterial composition and resuspending the bacterial composition in a formulation buffer.

In some embodiments, the formulation buffer comprises a lyoprotectant, a nutrient, a buffer, and an antioxidant. Example formulation buffers are described in PCT Publication No. WO 2018/081550, which is incorporated by reference herein in its entirety. In some embodiments, the disclosure provides a composition comprising a lyoprotectant, a nutrient, an antioxidant, and a buffer. In some embodiments, the formulation buffer comprises sucrose, yeast extract, L-cysteine, histidine, and magnesium chloride. In some embodiments, the formulation buffer comprises 7% sucrose, 0.1% yeast extract, 0.05% L-cysteine, 20 mM histidine, and 0.01% magnesium chloride. In some embodiments, the formulation buffer contains sodium metabisulfite. In some embodiments, the formulation buffer contains 0.05% sodium metabisulfite.

In some embodiments, the bacterial compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more bacteria. In some embodiments, the bacterial compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more bacteria per milliliter. It should be appreciated that some of the bacteria may not be viable. In some embodiments, the bacterial compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (cfus) of bacteria. In some embodiments, the bacterial compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (cfus) of bacteria per milliliter.

In some embodiments, the bacterial compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹ between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰ between 10³ and 10¹⁰, between 10⁴ and 10¹⁰, between 10⁵ and 10₁₀, between 10⁶ and 10¹⁰ between 10⁷ and 10¹⁰, between 10⁸ and 10¹⁰ between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per milliliter.

In some embodiments, the bacterial compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹ between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴ between 10² and 10⁴, between 10³ and 10⁴, between 10 and 10³, between 10² and 10³, or between 10 and 10² colony forming units of bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² colony forming units of bacteria per milliliter.

In some embodiments of the bacterial compositions provided herein, the composition includes at least 1×10⁸ colony forming units of bacteria per milliliter.

The methods described herein involve flash freezing a bacterial composition. As used herein, the term “flash freezing,” also known to as “snap freezing,” refers to a process by which the temperature of a bacterial composition is rapidly lowered to temperatures below −70° C., for example using liquid nitrogen or dry ice. In some embodiments, the flash freezing involves contacting the bacterial composition with a super-cooled surface. In some embodiments, the flash freezing involves contacting the bacterial composition with a receptacle containing liquid nitrogen or dry ice. In some embodiments, the flash freezing involves contacting the bacterial composition with liquid nitrogen. In some embodiments, the bacterial composition is applied to liquid nitrogen forming droplets. In some embodiments, the droplets of the bacterial composition are collected from the liquid nitrogen. In some embodiments, the droplets of the bacterial composition are collected from the liquid nitrogen and transferred to a receptacle for lyophilization (e.g., lyophilization vial).

Compositions that include bacterial strains can be lyophilized to preserve the bacterial strain. In some embodiments, the composition or the bacterial strains of the composition are lyophilized. Methods of lyophilizing compositions, including compositions comprising bacteria, are known in the art. See, e.g., U.S. Pat. Nos. 3,261,761; 4,205,132; PCT Publication Nos. WO 2014/029578, WO 2012/098358, WO2012/076665, and WO2012/088261, herein incorporated by reference in their entirety. However, finding conditions that allow for the lyophilization of certain bacteria, such as anaerobic bacteria has been challenging. See e.g., Peiren et al., Appl Microbol Biotechnol (2015) 99: 3559. It should be appreciated that in one aspect the methods of stabilization and preservation provided herein allow for the ability to generate compositions that allow for the manufacture of bacterial strains, in particular anaerobic bacterial strains. The methods described herein result in increased viability of lyophilized bacterial compositions.

Aspects of the disclosure provide methods of preparing a preserved bacterial composition involving flash freezing a bacterial composition and lyophilizing the flash frozen bacterial composition to produce a preserved bacterial composition. In general, lyophilization is a desiccation process to preserve a material, such as bacteria, involving freeze-drying. Water is removed from material by freezing the material and then placing the material under a vacuum, during which the ice undergoes sublimation. In some embodiments, the lyophilization cycle involves the steps of freezing, primary drying, and secondary drying. The term “temperature ramp rate” refers to the rate by which the temperature is adjusted between steps of the lyophilization cycle.

In some embodiments, the lyophilization cycle comprises a primary drying step and a secondary drying step, each of which involves subjecting the bacterial composition to a desired temperature and pressure.

In some embodiments, the primary drying step comprises subjecting the flash frozen bacterial composition to a temperature of about −30° C. to +10° C., −20° C. to 0° C., −15° C. to −5° C., or −12° C. to −7° C. In some embodiments, the primary drying step comprises subjecting the flash frozen bacterial composition to a temperature of about −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., +1° C., +2° C., +3° C., +4° C., +5° C., +6° C., +7° C., +8° C., +9° C., or +10° C. In some embodiments, the primary drying step comprises subjecting the flash frozen bacterial composition to a temperature of about −10° C.

In some embodiments, the primary drying step comprises subjecting the flash frozen bacterial composition to a pressure of about 50 mTorr to 90 mTorr, 60 mTorr to 80 mTorr, 65 mTorr to 75 mTorr, 60 mTorr to 70 mTorr, 55 mTorr to 75 mTorr, or 70 mTorr to 85 mTorr. In some embodiments, the primary drying step comprises subjecting the flash frozen bacterial composition to a pressure of about 50 mTorr, 51 mTorr, 52 mTorr, 53 mTorr, 54 mTorr, 55 mTorr, 56 mTorr, 57 mTorr, 58 mTorr, 59 mTorr, 60 mTorr, 61 mTorr, 62 mTorr, 63 mTorr, 64 mTorr, 65 mTorr, 66 mTorr, 67 mTorr, 68 mTorr, 69 mTorr, 70 mTorr, 71 mTorr, 72 mTorr, 73 mTorr, 74 mTorr, 75 mTorr, 76 mTorr, 77 mTorr, 78 mTorr, 79 mTorr, 80 mTorr, 81 mTorr, 82 mTorr, 83 mTorr, 84 mTorr, 85 mTorr, 86 mTorr, 87 mTorr, 88 mTorr, 89 mTorr, or 90 mTorr. In some embodiments, the primary drying step comprises subjecting the flash frozen bacterial composition to a pressure of about 70 mTorr.

In some embodiments, the primary drying step comprises subjecting the flash frozen bacterial composition to a temperature of about −10° C. and a pressure of about 70 mTorr.

In some embodiments, the secondary drying step comprises subjecting the flash frozen bacterial composition to a temperature of about 0° C. to +40° C., +10° C. to +30° C., +15° C. to +25° C., or +17° C. to +22° C. In some embodiments, the secondary drying step comprises subjecting the flash frozen bacterial composition to a temperature of about 0° C., +1° C., +2° C., +3° C., +4° C., +5° C., +6° C., +7° C., +8° C., +9° C., +10° C., +11° C., +12° C., +13° C., +14° C., +15° C., +16° C., +17° C., +18° C., +19° C., +20° C., +21° C., +22° C., +23° C., +24° C., +25° C., +26° C., +27° C., +28° C., +29° C., +30° C., +31° C., +32° C., +33° C., +34° C., +35° C., +36° C., +37° C., +38° C., +39° C., or +40° C. In some embodiments, the secondary drying step comprises subjecting the flash frozen bacterial composition to a temperature of about +20° C.

In some embodiments, the secondary drying step comprises subjecting the flash frozen bacterial composition to a pressure of about 50 mTorr to 90 mTorr, 60 mTorr to 80 mTorr, 65 mTorr to 75 mTorr, 60 mTorr to 70 mTorr, 55 mTorr to 75 mTorr, or 70 mTorr to 85 mTorr. In some embodiments, the secondary drying step comprises subjecting the flash frozen bacterial composition to a pressure of about 50 mTorr, 51 mTorr, 52 mTorr, 53 mTorr, 54 mTorr, 55 mTorr, 56 mTorr, 57 mTorr, 58 mTorr, 59 mTorr, 60 mTorr, 61 mTorr, 62 mTorr, 63 mTorr, 64 mTorr, 65 mTorr, 66 mTorr, 67 mTorr, 68 mTorr, 69 mTorr, 70 mTorr, 71 mTorr, 72 mTorr, 73 mTorr, 74 mTorr, 75 mTorr, 76 mTorr, 77 mTorr, 78 mTorr, 79 mTorr, 80 mTorr, 81 mTorr, 82 mTorr, 83 mTorr, 84 mTorr, 85 mTorr, 86 mTorr, 87 mTorr, 88 mTorr, 89 mTorr, or 90 mTorr. In some embodiments, the primary drying step comprises subjecting the flash frozen bacterial composition to a pressure of about 70 mTorr.

In some embodiments, the primary drying step comprises subjecting the flash frozen bacterial composition to a temperature of about +20° C. and a pressure of about 70 mTorr.

In some embodiments, the lyophilization cycle includes one or more steps having a temperature ramp rate between 0.5° C./min to 3° C./min. In some embodiments, the temperature ramp rate is 0.5° C./min, 0.6° C./min, 0.7° C./min, 0.8° C./min, 0.9° C./min, 1.0° C./min, 1.1° C./min, 1.2° C./min, 1.3° C./min, 1.4° C./min, 1.5° C./min, 1.6° C./min, 1.7° C./min, 1.8° C./min, 1.9° C./min, 2.0° C./min, 2.1° C./min, 2.2° C./min, 2.3° C./min, 2.4° C./min, 2.5° C./min, 2.6° C./min, 2.7° C./min, 2.8° C./min, 2.9° C./min, or 3.0° C./min. In some embodiments, the lyophilization cycle includes one or more steps having a temperature ramp rate of 1.0° C./min. In some embodiments, the lyophilization cycle includes one or more steps having a temperature ramp rate of 2.5° C./min.

In some embodiments, each of the steps of the lyophilization cycle have a temperature ramp rate between 0.5° C./min to 3° C./min. In some embodiments, the temperature ramp rate is 0.5° C./min, 0.6° C./min, 0.7° C./min, 0.8° C./min, 0.9° C./min, 1.0° C./min, 1.1° C./min, 1.2° C./min, 1.3° C./min, 1.4° C./min, 1.5° C./min, 1.6° C./min, 1.7° C./min, 1.8° C./min, 1.9° C./min, 2.0° C./min, 2.1° C./min, 2.2° C./min, 2.3° C./min, 2.4° C./min, 2.5° C./min, 2.6° C./min, 2.7° C./min, 2.8° C./min, 2.9° C./min, or 3.0° C./min. In some embodiments, each of the steps of the lyophilization cycle have a temperature ramp rate of 1.0° C./min. In some embodiments, each of the steps of the lyophilization cycle have a temperature ramp rate of 2.5° C./min.

In some embodiments, the preserved bacterial compositions are subjected to storage conditions for a period of time following lyophilization. In some embodiments, the preserved bacterial compositions are subjected a temperature of about −100° C. to −60° C., −90° C. to −70° C., −95° C. to −75° C., −85° C. to −75° C., −85° C. to −70° C., or −85° C. to −65° C. In some embodiments, the preserved bacterial compositions are subjected a temperature of about −100° C., −99° C., −98° C., −97° C., −96° C., −95° C., −94° C., −93° C., −92° C., −91° C., −90° C., −89° C., −88° C., −87° C., −86° C., −85° C., −84° C., −83° C., −82° C., −81° C., −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., or −60° C. following lyophilization. In some embodiments, the preserved bacterial compositions are subjected to a temperature of about −80° C. following lyophilization. In some embodiments, the preserved bacterial compositions are subjected to a temperature of about −80° C. for a period of time following lyophilization. In some embodiments, the period of time is at least 1 week, at least 2 weeks, at least 4 weeks, at least 2 months, at least 3 months, at least 6 months, or at least 1 year or more.

In some embodiments, the solid compositions that include bacterial strains provided herein may be formulated for administration as a pharmaceutical composition, e.g., by reconstitution of a lyophilized product. The term “pharmaceutical composition” as used herein means a product that results from the mixing or combining of a solid formulation provided herein and one or more pharmaceutically acceptable excipient.

An “acceptable” excipient refers to an excipient that must be compatible with the active ingredient (e.g., the bacterial strain) and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

In one aspect, the disclosure provides compositions that allow for the preservation of bacteria. In some embodiments, the bacteria are anaerobic bacteria. Compositions useful for the preservations of bacteria are also referred to herein as stabilizing compositions. A method for preparing a preserved bacterial composition, as used herein, refers to a method that promotes the viability of the bacteria therein and allows for the recovery of the bacteria following flash freezing and lyophilizing. The stabilization or preservation functionality of the composition can be assessed by comparing the number of viable bacteria (e.g., colony forming units) at two specific time points (e.g., at day 1 and at day 100). In some embodiments, the preservation functionality of the composition is assessed by comparing the number of viable bacteria (e.g., colony forming units) at a first time point to the number of viable bacteria (e.g., colony forming units) at a second time point.

In some embodiments, the preservation functionality of the composition is assessed by comparing the number of viable bacteria (e.g., colony forming units) prior to flash freezing to the number of viable bacteria (e.g., colony forming units) after flash freezing. In some embodiments, the preservation functionality of the composition is assessed by comparing the number of viable bacteria (e.g., colony forming units) prior to flash freezing to the number of viable bacteria (e.g., colony forming units) after lyophilizing. In some embodiments, the preservation functionality of the composition is assessed by comparing the number of viable bacteria (e.g., colony forming units) after flash freezing/prior to lyophilizing to the number of viable bacteria (e.g., colony forming units) after lyophilizing.

In some embodiments, the preservation functionality of the composition is assessed by comparing the number of viable bacteria (e.g., colony forming units) prior to flash freezing to the number of viable bacteria (e.g., colony forming units) after subjecting the composition to storage conditions for a period of time. In some embodiments, the preservation functionality of the composition is assessed by comparing the number of viable bacteria (e.g., colony forming units) after flash freezing/prior to lyophilizing to the number of viable bacteria (e.g., colony forming units) after subjecting the composition to storage conditions for a period of time. In some embodiments, the preservation functionality of the composition is assessed by comparing the number of viable bacteria (e.g., colony forming units) after lyophilizing to the number of viable bacteria (e.g., colony forming units) after subjecting the composition to storage conditions for a period of time.

If the number of colony forming units is the same or substantially the same at the two time points or over a time period, the composition is a perfect preserving method. A large decrease in the number of colony forming units between two time points or over a time period indicates that the method is not a good preserving composition.

In some embodiments, the methods provided herein allow for the recovery of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, up to 100% of the colony forming units over a period of time. In some embodiments, the period of time is at least 1 week, at least 2 weeks, at least 4 weeks, at least 2 months, at least 3 months, at least 6 months, or at least 1 year or more. In some embodiments, the percentage of recovered colony forming units or level of preservation is determined by comparing a number of colony forming units of bacteria (e.g., of a bacterial strain or total bacteria) at a first time point relative to the number of colony forming units of bacteria (e.g., of a bacterial strain or total bacteria) at a second time point over a period of time. For example, a 50% recovery or preservation of 50% of bacteria indicates that half of the bacteria remained viable over the period of time; and a 100% recovery indicates that all (or substantially all) bacteria remained viable over the period of time.

In some embodiments, the methods provided herein result in a level of viability of the preserved bacterial composition of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, up to 100% of the colony forming units over a period of time. In some embodiments, the period of time is at least 1 week, at least 2 weeks, at least 4 weeks, at least 2 months, at least 3 months, at least 6 months, or at least 1 year or more. In some embodiments, the level of viability is determined by comparing a number of colony forming units of bacteria (e.g., of a bacterial strain or total bacteria) at a first time point relative to the number of colony forming units of bacteria (e.g., of a bacterial strain or total bacteria) at a second time point over a period of time. For example, a 50% viability indicates that half of the bacteria remained viable over the period of time; and a 100% viability indicates that all (or substantially all) bacteria remained viable over the period of time.

In some embodiments, the methods provided herein result in preserved bacterial compositions having enhanced viability as compared to methods involving freezing and lyophilizing bacterial compositions in freeze-drying trays (e.g., GORE® Lyoguard® freeze-drying trays. In some embodiments, the methods described herein result in preserved bacterial compositions having viability that is enhanced by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to methods involving freezing and lyophilizing bacterial compositions in freeze-drying trays.

Strain 1 16S ribosomal RNA
Clostridium bolteae
SEQ ID NO: 1
ATGAGAGTTTGATCCTGGCTCAGGATGAACGCTGG
CGGCGTGCCTAACACATGCAAGTCGAACGAAGCAA
TTAAAATGAAGTTTTCGGATGGATTTTTGATTGAC
TGAGTGGCGGACGGGTGAGTAACGCGTGGATAACC
TGCCTCACACTGGGGGATAACAGTTAGAAATGACT
GCTAATACCGCATAAGCGCACAGTACCGCATGGTA
CGGTGTGAAAAACTCCGGTGGTGTGAGATGGATCC
GCGTCTGATTAGCCAGTTGGCGGGGTAACGGCCCA
CCAAAGCGACGATCAGTAGCCGACCTGAGAGGGTG
ACCGGCCACATTGGGACTGAGACACGGCCCAAACT
CCTACGGGAGGCAGCAGTGGGGAATATTGCACAAT
GGGCGAAAGCCTGATGCAGCGACGCCGCGTGAGTG
AAGAAGTATTTCGGTATGTAAAGCTCTATCAGCAG
GGAAGAAAATGACGGTACCTGACTAAGAAGCCCCG
GCTAACTACGTGCCAGCAGCCGCGGTAATACGTAG
GGGGCAAGCGTTATCCGGATTTACTGGGTGTAAAG
GGAGCGTAGACGGCGAAGCAAGTCTGAAGTGAAAA
CCCAGGGCTCAACCCTGGGACTGCTTTGGAAACTG
TTTTGCTAGAGTGTCGGAGAGGTAAGTGGAATTCC
TAGTGTAGCGGTGAAATGCGTAGATATTAGGAGGA
ACACCAGTGGCGAAGGCGGCTTACTGGACGATAAC
TGACGTTGAGGCTCGAAAGCGTGGGGAGCAAACAG
GATTAGATACCCTGGTAGTCCACGCCGTAAACGAT
GAATGCTAGGTGTTGGGGGGCAAAGCCCTTCGGTG
CCGTCGCAAACGCAGTAAGCATTCCACCTGGGGAG
TACGTTCGCAAGAATGAAACTCAAAGGAATTGACG
GGGACCCGCACAAGCGGTGGAGCATGTGGTTTAAT
TCGAAGCAACGCGAAGAACCTTACCAAGTCTTGAC
ATCCTCTTGACCGGCGTGTAACGGCGCCTTCCCT
TCGGGGCAAGAGAGACAGGTGGTGCATGG
TTGTCGTCAGCTCGTGTCGTGAGATGTTGGGTTAA
GTCCCGCAACGAGCGCAACCCTTATCCTTAGTAGC
CAGCAGGTAAAGCTGGGCACTCTAGGGAGACTGCC
AGGGATAACCTGGAGGAAGGTGGGGATGACGTCAA
ATCATCATGCCCCTTATGATTTGGGCTACACACGT
GCTACAATGGCGTAAACAAAGGGAAGCAAGACAGT
GATGTGGAGCAAATCCCAAAAATAACGTCCCAGTT
CGGACTGTAGTCTGCAACCCGACTACACGAAGCTG
GAATCGCTAGTAATCGCGAATCAGAATGTCGCGGT
GAATACGTTCCCGGGTCTTGTACACACCGCCCGTC
ACACCATGGGAGTCAGCAACGCCCGAAGTCAGTGA
CCCAACTCGCAAGAGAGGGAGCTGCCGAAGGCGGG
GCAGGTAACTGGGGTGAAGTCGTAACAAGGTAGCC
GTATCGGAAGGTGCGGCTGGATCACCTCCTTT
Strain 2 16S ribosomal RNA
Anaerotruncus colihominis
SEQ ID NO: 2
TCAAAGAGTTTGATCCTGGCTCAGGACGAACGCTG
GCGGCGCGCCTAACACATGCAAGTCGAACGGAGCT
TACGTTTTGAAGTTTTCGGATGGATGAATGTAAGC
TTAGTGGCGGACGGGTGAGTAACACGTGAGCAACC
TGCCTTTCAGAGGGGGATAACAGCCGGAAACGGCT
GCTAATACCGCATGATGTTGCGGGGGCACATGCCC
CTGCAACCAAAGGAGCAATCCGCTGAAAGATGGGC
TCGCGTCCGATTAGCCAGTTGGCGGGGTAACGGCC
CACCAAAGCGACGATCGGTAGCCGGACTGAGAGGT
TGAACGGCCACATTGGGACTGAGACACGGCCCAGA
CTCCTACGGGAGGCAGCAGTGGGGGATATTGCACA
ATGGGCGAAAGCCTGATGCAGCGACGCCGCGTGAG
GGAAGACGGTCTTCGGATTGTAAACCTCTGTCTTT
GGGGAAGAAAATGACGGTACCCAAAGAGGAAGCTC
CGGCTAACTACGTGCCAGCAGCCGCGGTAATACGT
AGGGAGCAAGCGTTGTCCGGAATTACTGGGTGTAA
AGGGAGCGTAGGCGGGATGGCAAGTAGAATGTTAA
ATCCATCGGCTCAACCGGTGGCTGCGTTCTAAACT
GCCGTTCTTGAGTGAAGTAGAGGCAGGCGGAATTC
CTAGTGTAGCGGTGAAATGCGTAGATATTAGGAGG
AACACCAGTGGCGAAGGCGGCCTGCTGGGCTTTAA
CTGACGCTGAGGCTCGAAAGCGTGGGGAGCAAACA
GGATTAGATACCCTGGTAGTCCACGCCGTAAACGA
TGATTACTAGGTGTGGGGGGACTGACCCCTTCCGT
GCCGCAGTTAACACAATAAGTAATCCACCTGGGGA
GTACGGCCGCAAGGTTGAAACTCAAAGGAATTGAC
GGGGGCCCGCACAAGCAGTGGAGTATGTGGTTTAA
TTCGAAGCAACGCGAAGAACCTTACCAGGTCTTGA
CATCGGATGCATAGCCTAGAGATAGGTGAAGCCCT
TCGGGGCATCCAGACAGGTGGTGCATGGTTGTCGT
CAGCTCGTGTCGTGAGATGTTGGGTTAAGTCCCGC
AACGAGCGCAACCCTTATTATTAGTTGCTACGCAA
GAGCACTCTAATGAGACTGCCGTTGACAAAACGGA
GGAAGGTGGGGATGACGTCAAATCATCATGCCCCT
TATGACCTGGGCTACACACGTACTACAATGGCACT
AAAACAGAGGGCGGCGACACCGCGAGGTGAAGCGA
ATCCCGAAAAAGTGTCTCAGTTCAGATTGCAGGCT
GCAACCCGCCTGCATGAAGTCGGAATTGCTAGTAA
TCGCGGATCAGCATGCCGCGGTGAATACGTTCCCG
GGCCTTGTACACACCGCCCGTCACACCATGGGAGT
CGGTAACACCCGAAGCCAGTAGCCTAACCGCAAGG
GGGGCGCTGTCGAAGGTGGGATTGATGACTGGGGT
GAAGTCGTAACAAGGTAGCCGTATCGGAAGGTGCG
GCTGGATCACCTCCTTT
Strain 3 16S ribosomal RNA
Ruminococcus torques
SEQ ID NO: 3
TACGAGAGTTTGATCCTGGCTCAGGATGAACGCTG
GCGGCGTGCCTAACACATGCAAGTCGAGCGAAGCG
CTGTTTTCAGAATCTTCGGAGGAAGAGGACAGTGA
CTGAGCGGCGGACGGGTGAGTAACGCGTGGGCAAC
CTGCCTCATACAGGGGGATAACAGTTAGAAATGAC
TGCTAATACCGCATAAGCGCACAGGACCGCATGGT
GTAGTGTGAAAAACTCCGGTGGTATGAGATGGACC
CGCGTCTGATTAGGTAGTTGGTGGGGTAAAGGCCT
ACCAAGCCGACGATCAGTAGCCGACCTGAGAGGGT
GACCGGCCACATTGGGACTGAGACACGGCCCAAAC
TCCTACGGGAGGCAGCAGTGGGGAATATTGCACAA
TGGGGGAAACCCTGATGCAGCGACGCCGCGTGAAG
GAAGAAGTATTTCGGTATGTAAACTTCTATCAGCA
GGGAAGAAAATGACGGTACCTGAGTAAGAAGCACC
GGCTAAATACGTGCCAGCAGCCGCGGTAATACGTA
TGGTGCAAGCGTTATCCGGATTTACTGGGTGTAAA
GGGAGCGTAGACGGATAGGCAAGTCTGGAGTGAAA
ACCCAGGGCTCAACCCTGGGACTGCTTTGGAAACT
GCAGATCTGGAGTGCCGGAGAGGTAAGCGGAATTC
CTAGTGTAGCGGTGAAATGCGTAGATATTAGGAGG
AACACCAGTGGCGAAGGCGGCTTACTGGACGGTGA
CTGACGTTGAGGCTCGAAAGCGTGGGGAGCAAACA
GGATTAGATACCCTGGTAGTCCACGCCGTAAACGA
TGACTACTAGGTGTCGGTGTGCAAAGCACATCGGT
GCCGCAGCAAACGCAATAAGTAGTCCACCTGGGGA
GTACGTTCGCAAGAATGAAACTCAAAGGAATTGAC
GGGGACCCGCACAAGCGGTGGAGCATGTGGTTTAA
TTCGAAGCAACGCGAAGAACCTTACCTGGTCTTGA
CATCCGGATGACGGGCGAGTAATGTCGCCGTCCCT
TCGGGGCGTCCGAGACAGGTGGTGCATGGTTGTCG
TCAGCTCGTGTCGTGAGATGTTGGGTTAAGTCCCG
CAACGAGCGCAACCCTTATCTTCAGTAGCCAGCAT
ATAAGGTGGGCACTCTGGAGAGACTGCCAGGGAGA
ACCTGGAGGAAGGTGGGGATGACGTCAAATCATCA
TGCCCCTTATGGCCAGGGCTACACACGTGCTACAA
TGGCGTAAACAAAGGGAAGCGAGAGGGTGACCTGG
AGCGAATCCCAAAAATAACGTCTCAGTTCGGATTG
TAGTCTGCAACTCGACTACATGAAGCTGGAATCGC
TAGTAATCGCGGATCAGCATGCCGCGGTGAATACG
TTCCCGGGTCTTGTACACACCGCCCGTCACACCAT
GGGAGTCAGTAACGCCCGAAGCCAGTGACCCAACC
TTAGAGGAGGGAGCTGTCGAAGGCGGGACGGATAA
CTGGGGTGAAGTCGTAACAAGGTAGCCGTATCGGA
AGGTGCGGCTGGATCACCTCCTTT
Strain 4 16S ribosomal RNA
Clostridium symbiosum
SEQ ID NO: 4
ATGAGAGTTTGATCCTGGCTCAGGATGAACGCTGG
CGGCGTGCCTAACACATGCAAGTCGAACGAAGCGA
TTTAACGGAAGTTTTCGGATGGAAGTTGAATTGAC
TGAGTGGCGGACGGGTGAGTAACGCGTGGGTAACC
TGCCTTGTACTGGGGGACAACAGTTAGAAATGACT
GCTAATACCGCATAAGCGCACAGTATCGCATGATA
CAGTGTGAAAAACTCCGGTGGTACAAGATGGACCC
GCGTCTGATTAGCTAGTTGGTAAGGTAACGGCTTA
CCAAGGCGACGATCAGTAGCCGACCTGAGAGGGTG
ACCGGCCACATTGGGACTGAGACACGGCCCAAACT
CCTACGGGAGGCAGCAGTGGGGAATATTGCACAAT
GGGCGAAAGCCTGATGCAGCGACGCCGCGTGAGTG
AAGAAGTATTTCGGTATGTAAAGCTCTATCAGCAG
GGAAGAAAATGACGGTACCTGACTAAGAAGCCCCG
GCTAACTACGTGCCAGCAGCCGCGGTAATACGTAG
GGGGCAAGCGTTATCCGGATTTACTGGGTGTAAAG
GGAGCGTAGACGGTAAAGCAAGTCTGAAGTGAAAG
CCCGCGGCTCAACTGCGGGACTGCTTTGGAAACTG
TTTAACTGGAGTGTCGGAGAGGTAAGTGGAATTCC
TAGTGTAGCGGTGAAATGCGTAGATATTAGGAGGA
ACACCAGTGGCGAAGGCGACTTACTGGACGATAAC
TGACGTTGAGGCTCGAAAGCGTGGGGAGCAAACAG
GATTAGATACCCTGGTAGTCCACGCCGTAAACGAT
GAATACTAGGTGTTGGGGAGCAAAGCTCTTCGGTG
CCGTCGCAAACGCAGTAAGTATTCCACCTGGGGAG
TACGTTCGCAAGAATGAAACTCAAAGGAATTGACG
GGGACCCGCACAAGCGGTGGAGCATGTGGTTTAAT
TCGAAGCAACGCGAAGAACCTTACCAGGTCTTGAC
ATCGATCCGACGGGGGAGTAACGTCCCCTTCCCTT
CGGGGCGGAGAAGACAGGTGGTGCATGGTTGTCGT
CAGCTCGTGTCGTGAGATGTTGGGTTAAGTCCCGC
AACGAGCGCAACCCTTATTCTAAGTAGCCAGCGGT
TCGGCCGGGAACTCTTGGGAGACTGCCAGGGATAA
CCTGGAGGAAGGTGGGGATGACGTCAAATCATCAT
GCCCCTTATGATCTGGGCTACACACGTGCTACAAT
GGCGTAAACAAAGAGAAGCAAGACCGCGAGGTGGA
GCAAATCTCAAAAATAACGTCTCAGTTCGGACTGC
AGGCTGCAACTCGCCTGCACGAAGCTGGAATCGCT
AGTAATCGCGAATCAGAATGTCGCGGTGAATACGT
TCCCGGGTCTTGTACACACCGCCCGTCACACCATG
GGAGTCAGTAACGCCCGAAGTCAGTGACCCAACCG
CAAGGAGGGAGCTGCCGAAGGCGGGACCGATAACT
GGGGTGAAGTCGTAACAAGGTAGCCGTATCGGAAG
GTGCGGCTGGATCACCTCCTTT
Strain 5 16S ribosomal RNA
Blautia producta
SEQ ID NO: 5
ATCAGAGAGTTTGATCCTGGCTCAGGATGAACGCT
GGCGGCGTGCTTAACACATGCAAGTCGAGCGAAGC
ACTTAAGTGGATCTCTTCGGATTGAAGCTTATTTG
ACTGAGCGGCGGACGGGTGAGTAACGCGTGGGTAA
CCTGCCTCATACAGGGGGATAACAGTTAGAAATGG
CTGCTAATACCGCATAAGCGCACAGGACCGCATGG
TCTGGTGTGAAAAACTCCGGTGGTATGAGATGGAC
CCGCGTCTGATTAGCTAGTTGGAGGGGTAACGGCC
CACCAAGGCGACGATCAGTAGCCGGCCTGAGAGGG
TGAACGGCCACATTGGGACTGAGACACGGCCCAGA
CTCCTACGGGAGGCAGCAGTGGGGAATATTGCACA
ATGGGGGAAACCCTGATGCAGCGACGCCGCGTGAA
GGAAGAAGTATCTCGGTATGTAAACTTCTATCAGC
AGGGAAGAAAATGACGGTACCTGACTAAGAAGCCC
CGGCTAACTACGTGCCAGCAGCCGCGGTAATACGT
AGGGGGCAAGCGTTATCCGGATTTACTGGGTGTAA
AGGGAGCGTAGACGGAAGAGCAAGTCTGATGTGAA
AGGCTGGGGCTTAACCCCAGGACTGCATTGGAAAC
TGTTTTTCTAGAGTGCCGGAGAGGTAAGCGGAATT
CCTAGTGTAGCGGTGAAATGCGTAGATATTAGGAG
GAACACCAGTGGCGAAGGCGGCTTACTGGACGGTA
ACTGACGTTGAGGCTCGAAAGCGTGGGGAGCAAAC
AGGATTAGATACCCTGGTAGTCCACGCCGTAAACG
ATGAATACTAGGTGTCGGGTGGCAAAGCCATTCGG
TGCCGCAGCAAACGCAATAAGTATTCCACCTGGGG
AGTACGTTCGCAAGAATGAAACTCAAAGGAATTGA
CGGGGACCCGCACAAGCGGTGGAGCATGTGGTTTA
ATTCGAAGCAACGCGAAGAACCTTACCAAGTCTTG
ACATCCCTCTGACCGGCCCGTAACGGGGCCTTCCC
TTCGGGGCAGAGGAGACAGGTGGTGCATGGTTGTC
GTCAGCTCGTGTCGTGAGATGTTGGGTTAAGTCCC
GCAACGAGCGCAACCCCTATCCTTAGTAGCCAGCA
GGTGAAGCTGGGCACTCTAGGGAGACTGCCGGGGA
TAACCCGGAGGAAGGCGGGGACGACGTCAAATCAT
CATGCCCCTTATGATTTGGGCTACACACGTGCTAC
AATGGCGTAAACAAAGGGAAGCGAGACAGCGATGT
TGAGCAAATCCCAAAAATAACGTCCCAGTTCGGAC
TGCAGTCTGCAACTCGACTGCACGAAGCTGGAATC
GCTAGTAATCGCGAATCAGAATGTCGCGGTGAATA
CGTTCCCGGGTCTTGTACACACCGCCCGTCACACC
ATGGGAGTCAGTAACGCCCGAAGTCAGTGACCCAA
CCTTACAGGAGGGAGCTGCCGAAGGCGGGACCGAT
AACTGGGGTGAAGTCGTAACAAGGTAGCCGTATCG
GAAGGTGCGGCTGGATCACCTCCTTT
Strain 6 16S ribosomal RNA
Dorea Longicatena
SEQ ID NO: 6
AACGAGAGTTTGATCCTGGCTCAGGATGAACGCTG
GCGGCGTGCTTAACACATGCAAGTCGAGCGAAGCA
CTTAAGTTTGATTCTTCGGATGAAGACTTTTGTGA
CTGAGCGGCGGACGGGTGAGTAACGCGTGGGTAAC
CTGCCTCATACAGGGGGATAACAGTTAGAAATGAC
TGCTAATACCGCATAAGACCACGGTACCGCATGGT
ACAGTGGTAAAAACTCCGGTGGTATGAGATGGACC
CGCGTCTGATTAGGTAGTTGGTGGGGTAACGGCCT
ACCAAGCCGACGATCAGTAGCCGACCTGAGAGGGT
GACCGGCCACATTGGGACTGAGACACGGCCCAGAC
TCCTACGGGAGGCAGCAGTGGGGAATATTGCACAA
TGGAGGAAACTCTGATGCAGCGACGCCGCGTGAAG
GATGAAGTATTTCGGTATGTAAACTTCTATCAGCA
GGGAAGAAAATGACGGTACCTGACTAAGAAGCCCC
GGCTAACTACGTGCCAGCAGCCGCGGTAATACGTA
GGGGGCAAGCGTTATCCGGATTTACTGGGTGTAAA
GGGAGCGTAGACGGCACGGCAAGCCAGATGTGAAA
GCCCGGGGCTCAACCCCGGGACTGCATTTGGAACT
GCTGAGCTAGAGTGTCGGAGAGGCAAGTGGAATTC
CTAGTGTAGCGGTGAAATGCGTAGATATTAGGAGG
AACACCAGTGGCGAAGGCGGCTTGCTGGACGATGA
CTGACGTTGAGGCTCGAAAGCGTGGGGAGCAAACA
GGATTAGATACCCTGGTAGTCCACGCCGTAAACGA
TGACTGCTAGGTGTCGGGTGGCAAAGCCATTCGGT
GCCGCAGCTAACGCAATAAGCAGTCCACCTGGGGA
GTACGTTCGCAAGAATGAAACTCAAAGGAATTGAC
GGGGACCCGCACAAGCGGTGGAGCATGTGGTTTAA
TTCGAAGCAACGCGAAGAACCTTACCTGATCTTGA
CATCCCGATGACCGCTTCGTAATGGAAGCTTTTCT
TCGGAACATCGGTGACAGGTGGTGCATGGTTGTCG
TCAGCTCGTGTCGTGAGATGTTGGGTTAAGTCCCG
CAACGAGCGCAACCCCTATCTTCAGTAGCCAGCAG
GTTAAGCTGGGCACTCTGGAGAGACTGCCAGGGAT
AACCTGGAGGAAGGTGGGGATGACGTCAAATCATC
ATGCCCCTTATGACCAGGGCTACACACGTGCTACA
ATGGCGTAAACAAAGAGAAGCGAACTCGCGAGGGT
AAGCAAATCTCAAAAATAACGTCTCAGTTCGGATT
GTAGTCTGCAACTCGACTACATGAAGCTGGAATCG
CTAGTAATCGCAGATCAGAATGCTGCGGTGAATAC
GTTCCCGGGTCTTGTACACACCGCCCGTCACACCA
TGGGAGTCAGTAACGCCCGAAGTCAGTGACCCAAC
CGTAAGGAGGGAGCTGCCGAAGGTGGGACCGATAA
CTGGGGTGAAGTCGTAACAAGGTAGCCGTATCGGA
AGGTGCGGCTGGATCACCTCCTTT
Strain 7 16S ribosomal RNA
Erysipelotrichaceae bacterium
SEQ ID NO: 7
ATGGAGAGTTTGATCCTGGCTCAGGATGAACGCTG
GCGGCATGCCTAATACATGCAAGTCGAACGAAGTT
TCGAGGAAGCTTGCTTCCAAAGAGACTTAGTGGCG
AACGGGTGAGTAACACGTAGGTAACCTGCCCATGT
GTCCGGGATAACTGCTGGAAACGGTAGCTAAAACC
GGATAGGTATACAGAGCGCATGCTCAGTATATTAA
AGCGCCCATCAAGGCGTGAACATGGATGGACCTGC
GGCGCATTAGCTAGTTGGTGAGGTAACGGCCCACC
AAGGCGATGATGCGTAGCCGGCCTGAGAGGGTAAA
CGGCCACATTGGGACTGAGACACGGCCCAAACTCC
TACGGGAGGCAGCAGTAGGGAATTTTCGTCAATGG
GGGAAACCCTGAACGAGCAATGCCGCGTGAGTGAA
GAAGGTCTTCGGATCGTAAAGCTCTGTTGTAAGTG
AAGAACGGCTCATAGAGGAAATGCTATGGGAGTGA
CGGTAGCTTACCAGAAAGCCACGGCTAACTACGTG
CCAGCAGCCGCGGTAATACGTAGGTGGCAAGCGTT
ATCCGGAATCATTGGGCGTAAAGGGTGCGTAGGTG
GCGTACTAAGTCTGTAGTAAAAGGCAATGGCTCAA
CCATTGTAAGCTATGGAAACTGGTATGCTGGAGTG
CAGAAGAGGGCGATGGAATTCCATGTGTAGCGGTA
AAATGCGTAGATATATGGAGGAACACCAGTGGCGA
AGGCGGTCGCCTGGTCTGTAACTGACACTGAGGCA
CGAAAGCGTGGGGAGCAAATAGGATTAGATACCCT
AGTAGTCCACGCCGTAAACGATGAGAACTAAGTGT
TGGAGGAATTCAGTGCTGCAGTTAACGCAATAAGT
TCTCCGCCTGGGGAGTATGCACGCAAGTGTGAAAC
TCAAAGGAATTGACGGGGGCCCGCACAAGCGGTGG
AGTATGTGGTTTAATTCGAAGCAACGCGAAGAACC
TTACCAGGCCTTGACATGGAAACAAATACCCTAGA
GATAGGGGGATAATTATGGATCACACAGGTGGTGC
ATGGTTGTCGTCAGCTCGTGTCGTGAGATGTTGGG
TTAAGTCCCGCAACGAGCGCAACCCTTGTCGCATG
TTACCAGCATCAAGTTGGGGACTCATGCGAGACTG
CCGGTGACAAACCGGAGGAAGGTGGGGATGACGTC
AAATCATCATGCCCCTTATGGCCTGGGCTACACAC
GTACTACAATGGCGGCCACAAAGAGCAGCGACACA
GTGATGTGAAGCGAATCTCATAAAGGTCGTCTCAG
TTCGGATTGAAGTCTGCAACTCGACTTCATGAAGT
CGGAATCGCTAGTAATCGCAGATCAGCATGCTGCG
GTGAATACGTTCTCGGGCCTTGTACACACCGCCCG
TCAAACCATGGGAGTCAGTAATACCCGAAGCCGGT
GGCATAACCGTAAGGAGTGAGCCGTCGAAGGTAGG
ACCGATGACTGGGGTTAAGTCGTAACAAGGTATCC
CTACGGGAACGTGGGGATGGATCACCTCCTTT
Strain 8 16S ribosomal RNA
Subdoligramilum spp
SEQ ID NO: 8
TATTGAGAGTTTGATCCTGGCTCAGGATGAACGCT
GGCGGCGTGCTTAACACATGCAAGTCGAACGGGGT
GCTCATGACGGAGGATTCGTCCAACGGATTGAGTT
ACCTAGTGGCGGACGGGTGAGTAACGCGTGAGGAA
CCTGCCTTGGAGAGGGGAATAACACTCCGAAAGGA
GTGCTAATACCGCATGATGCAGTTGGGTCGCATGG
CTCTGACTGCCAAAGATTTATCGCTCTGAGATGGC
CTCGCGTCTGATTAGCTAGTAGGCGGGGTAACGGC
CCACCTAGGCGACGATCAGTAGCCGGACTGAGAGG
TTGACCGGCCACATTGGGACTGAGACACGGCCCAG
ACTCCTACGGGAGGCAGCAGTGGGGAATATTGGGC
AATGGGCGCAAGCCTGACCCAGCAACGCCGCGTGA
AGGAAGAAGGCTTTCGGGTTGTAAACTTCTTTTGT
CGGGGACGAAACAAATGACGGTACCCGACGAATAA
GCCACGGCTAACTACGTGCCAGCAGCCGCGGTAAT
ACGTAGGTGGCAAGCGTTATCCGGATTTACTGGGT
GTAAAGGGCGTGTAGGCGGGATTGCAAGTCAGATG
TGAAAACTGGGGGCTCAACCTCCAGCCTGCATTTG
AAACTGTAGTTCTTGAGTGCTGGAGAGGCAATCGG
AATTCCGTGTGTAGCGGTGAAATGCGTAGATATAC
GGAGGAACACCAGTGGCGAAGGCGGATTGCTGGAC
AGTAACTGACGCTGAGGCGCGAAAGCGTGGGGAGC
AAACAGGATTAGATACCCTGGTAGTCCACGCCGTA
AACGATGGATACTAGGTGTGGGGGGTCTGACCCCC
TCCGTGCCGCAGTTAACACAATAAGTATCCCACCT
GGGGAGTACGATCGCAAGGTTGAAACTCAAAGGAA
TTGACGGGGGCCCGCACAAGCGGTGGAGTATGTGG
TTTAATTCGAAGCAACGCGAAGAACCTTACCAGGG
CTTGACATCCCACTAACGAAGCAGAGATGCATTAG
GTGCCCTTCGGGGAAAGTGGAGACAGGTGGTGCAT
GGTTGTCGTCAGCTCGTGTCGTGAGATGTTGGGTT
AAGTCCCGCAACGAGCGCAACCCTTATTGTTAGTT
GCTACGCAAGAGCACTCTAGCGAGACTGCCGTTGA
CAAAACGGAGGAAGGTGGGGACGACGTCAAATCAT
CATGCCCCTTATGTCCTGGGCCACACACGTACTAC
AATGGTGGTTAACAGAGGGAGGCAATACCGCGAGG
TGGAGCAAATCCCTAAAAGCCATCCCAGTTCGGAT
TGCAGGCTGAAACCCGCCTGTATGAAGTTGGAATC
GCTAGTAATCGCGGATCAGCATGCCGCGGTGAATA
CGTTCCCGGGCCTTGTACACACCGCCCGTCACACC
ATGAGAGTCGGGAACACCCGAAGTCCGTAGCCTAA
CCGCAAGGAGGGCGCGGCCGAAGGTGGGTTCGATA
ATTGGGGTGAAGTCGTAACAAGGTAGCCGTATCGG
AAGGTGCGGCTGGATCACCTCCTTT

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### Example

Studies were performed to test flash freezing liquid droplets of formulated culture before lyophilization. Testing demonstrated an improved viability of bacterial strains after freezing and lyophilizing when compared to bacterial strains that were frozen and lyophilized in freeze-drying trays (e.g., GORE® Lyoguard® freeze-drying trays). Along with the improvements in viability, the ability to freeze and store frozen pellets allows greater flexibility with the production of preserved bacterial products.

Bacterial cultures were inoculated in growth media and incubated anaerobically at 37° C. Once the optical density (OD) exceeded a target threshold based on the strain to be tested, the culture was spun down and resuspended in formulation buffer. The formulation buffer used has 70 g/L sucrose, 1 g/L yeast extract, 0.5 g/L L-cysteine, 20 mM histidine, and 0.1 g/L magnesium chloride. The formulation buffers for VE303-1, VE303-2 and VE303-6 also contained 0.5 g/L sodium metabisulfite. The formulation buffer used for the traditional freeze-drying method was identical, except that it did not contain magnesium chloride. The culture was spun down a second time, the supernatant discarded, and suspended again in formulation buffer. Droplets of the culture were flash frozen by adding them to a liquid nitrogen bath using a 1 mL pipette. The frozen droplets were collected using a sieve and aliquoted to lyophilization vials that were on dry ice. The vials were transferred to a lyophilizer with a shelf temperature of −50° C. All vials were held at −50° C. for 4 hours. Primary drying was performed at −10° C. and 70 mTorr. Secondary drying was performed at +20° C. and 70 mTorr. The vials were then removed from the lyophilizer and stored at −80° C. until they were able to be tested.

For each bacterial strain, a harvest sample, freeze-thaw sample (data not shown), and post-lyophilization samples for each of the two methods were plated to determine viability of the bacterial strain at each stage of the process. Plates were made by producing serial dilutions for all samples in reduced phosphate buffered saline (PBS). A 100 μL aliquot of the sample was mixed in 9004, of 1×PBS. Serial dilutions were performed by mixing 100 μL from the previous dilution in 9004, of PBS. This was performed to generate dilutions from 10⁻¹ through 10⁻⁷. The 10⁻⁵, 10⁻⁶, and 10⁻⁷ dilutions were used to plate 1004, on a chocolate agar plate. The plated dilutions were spread using sterile beads and incubated anaerobically at 37° C. for >48 hours. The colonies on each plates were enumerated to determine the viability of each sample. For the post-lyophilization sample, 0.1 grams of material was rehydrated in PBS prior to dilution and plating to determine viability.

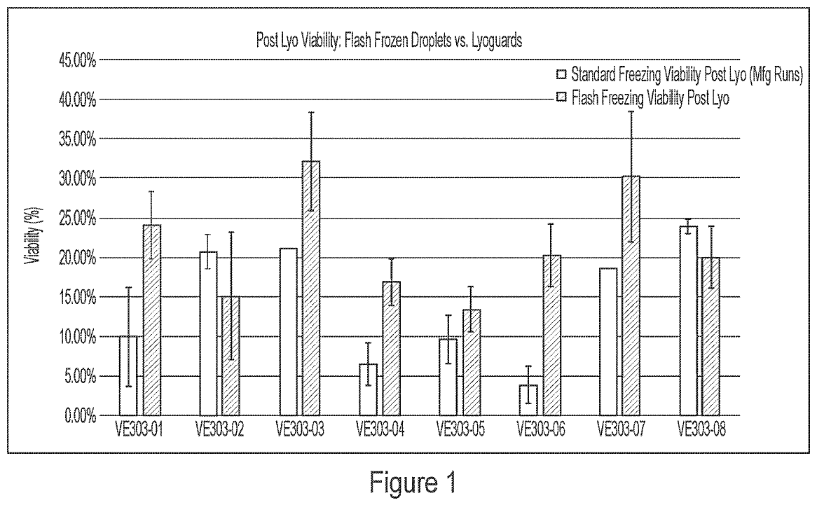

The post-lyophilization viability of each bacterial strain was compared to the post-lyophilization viability of the respective bacterial strain using traditional methods of freezing bacterial culture in a freeze-drying tray (e.g., GORE® Lyoguard® freeze-drying trays) (FIG. 1). Bacterial strain VE303-06 demonstrated the highest improvement in viability (increased from 3.9% using a freeze-drying tray to 20% using the methods described herein).

What is claimed is:

1. A method of preparing a preserved bacterial composition, comprising
flash freezing a bacterial composition, and
lyophilizing the flash frozen bacterial composition to produce a preserved bacterial composition.

2. The method of claim 1, wherein the bacterial composition comprises one or more bacterial strains.

3. The method of claim 1 or 2, wherein the bacterial composition comprises one or more anaerobic bacterial strains.

4. The method of claim 3, wherein the anaerobic bacterial strains are strict anaerobic bacteria.

5. The method of any one of claims 1-4, wherein the bacterial composition comprises one or more bacterial strains belong to the class Clostridia.

6. The method of claim 5, wherein one or more bacterial strains belong to the family Clostridiaceae.

7. The method of claim 6, wherein the bacteria comprise one or more bacterial strains belonging to the genus Clostridium.

8. The method of any one of claims 1-7, wherein the bacterial composition comprises one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavinofractor plautii.

9. The method of any one of claims 1-8, wherein the bacterial composition comprises one or more bacterial strains comprising 16S rDNA sequences having at least 97% sequence identity with the nucleic acid sequences selected from the group consisting of SEQ ID NOs: 1-8.

10. The method of any one of claims 1-9, further comprising washing the bacterial composition and resuspending the bacterial composition in a formulation buffer.

11. The method of any one of claims 1-10, wherein the flash freezing is performed by contacting the bacterial composition with a super-cooled surface.

12. The method of any one of claims 1-10, wherein the flash freezing is performed by contacting the bacterial composition with liquid nitrogen.

13. The method of any one of claims 1-12, wherein the bacterial composition has a symmetrical shape.

14. The method of claim 13, wherein the symmetrical shape is a symmetrical frozen droplet.

15. The method of any one of claims 1-14, further comprising subjecting the preserved bacterial composition to a temperature of −80° C.

16. The method of any one of claims 1-15, wherein the lyophilizing comprises a primary drying step and a secondary drying step.

17. The method of claim 16, wherein the primary drying step comprises subjecting the flash frozen bacterial composition to a temperature of −10° C. and under a pressure of 70 mTorr.

18. The method of claim 16 or 17, wherein the secondary drying step comprises subjecting the flash frozen bacterial composition to a temperature of +20° C. and under a pressure of 70 mTorr.

19. The method of any one of claims 1-18, further comprising culturing the bacterial composition.

20. The method of any one of claims 1-19, further comprising determining a level of viability in the preserved bacterial composition after lyophilizing.

21. The method of claim 20, wherein the level of viability in the preserved bacterial composition is at least 10%, at least 15%, at least 20%, at least 25%, at least 30%, at least 35%, or at least 40% of colony forming units of the bacteria over a period of time.

22. The method of claim 21, wherein the period of time is at least 1 week, at least 2 weeks, at least 4 weeks, at least 2 months, at least 3 months, at least 6 months, or at least 1 year or more.
